(19) United States
(12) Patent Application Publication
(10) Pub. No.: US 2003/0086952 A1
(43) Pub. Date: May 8, 2003

(54) Highhly concentrated emulsions of organic compounds and methos of use

(76) Inventors: Thomas C. Miller, Davis, CA (US); Ralph W. Emerson, Davis, CA (US)

Correspondence Address: RAE-VENTER LAW GROUP, P.C., P.O. BOX 1898, MONTEREY, CA, 93942-1898, US

(21) Appl. No.: 10/177,037

(22) Filed: Jun. 21, 2002

Related U.S. Application Data
(60) Provisional application No. 60/300,153, filed on Jun. 21, 2001.

(51) Int. Cl. A61K 7/50; A01N 25/00; C11D 1/00; C09K 3/00

(52) U.S. Cl. 424/401; 424/405; 510/130; 516/53

(57) ABSTRACT

Method and compositions are provided for preparing concentrated emulsions of organic compounds which are recalcitrant to aqueous solubilization. The compositions are useful for increasing the practical utility of aqueous-recalcitrant compounds for a variety of established functions. The highly concentrated emulsions are prepared through a two step mixing process. The invention is exemplified by preparation of stable saponin-based emulsions containing greater than 50% by weight benzyl benzoate, benzyl salicylate, cedryl acetate, D- or L-limonene, cinnamic aldehyde, and vanillin.

### CROSS-REFERENCE TO RELATED APPLICATIONS

The application claims benefit of the filing date of provisional application No. 60/300,153 filed Jun. 21, 2001, which disclosure is incorporated herein by reference.

### INTRODUCTION

1. Technical Field

The invention relates to methods for obtaining highly concentrated emulsions of benzyl propenoids and terpenes and the compositions so produced. The invention is exemplified by preparation of stable saponin-based emulsions containing greater than 50% by weight of benzyl benzoate, benzyl salicylate, cedryl acetate, D- or L-limonene, cinnamic aldehyde and vanillin.

2. Background

Recent years have seen rapid advances in many areas of practical chemistry, including agriculture, foodstuffs, cosmetics, pharmaceuticals, and pest control. As the universe of available compounds and their uses expands, certain practical limitations based on the physical properties of certain compounds and classes of compounds have come to light. It is with some regularity that a chosen compound for a particular use may function perfectly in laboratory trials, but remain tanzalizingly beyond the reach of commercial or industrial utility because of its physical properties. These properties may manifest themselves as prohibitively high production cost, inordinately dangerous or toxic properties, scarcity of material, or solubility constraints. In the latter case, for example, it may be that a particular oily or hydrophobic compound is not practical for commercial use because it can not be concentrated in an aqueous solution to a satisfactory degree. Attempts may be made to circumvent such problems by providing surfactants or emulsifiers to a compound, which will generally increase aqueous solubility of a hydrophobic compound. Often such emulsifiers are able to increase solubility only marginally, or, alternatively, may be synthetic compounds with inherent toxic properties, thus further limiting the use of the emulsified compound. It therefore is of interest to develop methods to prepare concentrated emulsions of compounds that are hydrophobic in nature, that are efficient and safe for industrial and agricultural uses.

### Relevant Literature

Art-recognized methods and compositions which comprise aqueous-recalcitrant compounds, and which can be improved by the invention described herein, include cosmetic preparations (see, e.g., U.S. Pat. Nos. 5,833,999 and 5,198,218), fragrance preparations (see, e.g., U.S. Pat. No. 5,916,528), food additives (see, e.g., U.S. Pat. Nos. 5,589,158 and 5,895,657), insect repellant preparations (see, e.g., U.S. Pat. No. 4,427,700), and pest control agents (see, e.g., U.S. Pat. Nos. 6,117,440 and 5,839,224). U.S. Pat. No. 4,379,168 discloses a pesticide composition made of D-limonene along with surfactants and water. U.S. Pat. No. 5,602,090 discloses surfactant based compositions with D-limonene. U.S. Pat. No. 6,130,253 discloses terpene based pesticide treatments. U.S. Pat. No. 6,207,705 discloses biopesticides made from glucosinolates and monoterpenoids. U.S. Pat. No. 5,736,584 discloses an insect repellent made of D-limonene and a octylphenoxy polyethoxy ethanol surfactant. U.S. Pat. No. 5,723,423 discloses compositions that include a surfactant and a terpenoid for cleaning oil-contaminated substrates.

### SUMMARY OF THE INVENTION

Provided are methods for emulsifying an organic compound in an aqueous solvent using low concentrations of surfactant to produce a useable emulsion containing a high concentration of the organic compound, and the emulsions produced by the method. The method includes the steps of combining the organic compound, with fixed ratios of an emulsifier such as a saponin compound, and mixing these components together with an appropriate solvent for a time sufficient to form a stable emulsion of the organic compound. Once a stable emulsion has formed, additional organic compound can be added to increase the concentration of the organic compound in the emulsion. The invention finds use in the preparation of stable emulsions of organic compounds that are recalcitrant to aqueous solubilization.

### DESCRIPTION OF THE SPECIFIC EMBODIMENTS

The present invention is directed to methods of emulsifying an organic compound which is recalcitrant to aqueous solubilization, including benzyl propenoids and terpenes, such as benzyl benzoate and benzyl salicylate, cedryl acetate, terpinyl acetate, vanillin and D-limonene and to compositions so prepared. As used herein, the term “recalcitrant to aqueous solubilization” includes the property of some compounds to be partially soluble or insoluble in aqueous (e.g., water-based) solvents. The property of being recalcitrant to aqueous solubilization may be caused by hydrophobic properties of the compound to be solubilized. Such a property may also be a “conditional property”, e.g., recalcitrance to aqueous solubilization only manifests in the presence of (or by reacting with) certain other components or solutes of a particular composition. For clarity, the term “compound which is recalcitrant to aqueous solubilization” is used interchangeably with the term “aqueous-recalcitrant compound.” To prepare the emulsions containing high concentrations of an organic compound, the organic compound is mixed with an emulsifying agent such as saponin in an aqueous solvent, generally water, while maintaining a ratio of at least 50% by weight of the organic compound, less than 10% by weight of the emulsifying agent and the balance of solvent. The mixture is agitated, generally by mechanical mixing, until an emulsion has formed, at which time optionally additional organic compound can be added to the emulsion if it is desired to further increase the concentration of organic compound in the emulsion. Emulsions of 60%, 70%, 80% and up to 90% can be prepared in this way, depending upon the organic compound. Preferred compounds that can be emulsified using this procedure include terpenes. As used herein, the art recognized term “terpene” includes hydrocarbon compounds which may be of biological origin and which have carbon skeletons formally derived from isoprene (CH2═C(CH3)CHCH2). The terpene compounds of the invention include the monoterpenes which comprise 10 carbons (C₁₀). Hemiterpenes, sesquiterpenes, diterpenes, are also within the scope of the invention. Examples of terpene compounds include the terpinenes, erpinolenes, limonenes, pinenes and mixtures thereof. Particularly preferred terpenes include D-limonene (C₁₀H₁₆; CAS Reg. No. 138-86-3). D-limonene is also referred to as dl-limonene, wherein “dl” signifies “dextro-levo.” The compound D-limonene is, like many terpenes, generally hydrophobic and recalcitrant to being dissolved in aqueous solutions. The structure of D-limonene is set forth below.

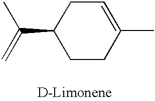

As used herein, the art-recognized term “emulsify” includes the act or process of facilitating a stable mixture or dispersion of otherwise immiscible compounds or liquids (e.g., an oily liquid and an aqueous liquid) through the use of an emulsifier or emulsifying compound. As further used herein, the art-recognized terms “emulsifier” and “emulsifying compound” include a compound or a mixture of compounds which comprises surface-active molecules and which can stabilize a mixture or dispersion of otherwise immiscible compounds or liquids (e.g., an emulsion). Generally emulsifiers act either by coating one or more of the components of the mixture to prevent coalescing and/or alter the surface tension at the interface of suspended droplets. “Synthetic emulsifiers” include emulsifier molecules which are designed and produced by standard chemistry methodology. “Organic emulsifiers” include emulsifier molecules which may be identified and/or produced from an organism (e.g., plant material, animal material). In the present invention, the preferred emulsifiers are extracts from the Yucca plant that contain saponin, generally about 5% to 15% saponin. Emulsification properties are obtainable with at little as 3% saponin, however, formulations comprising between 7-14% are preferred.

As used herein, the art-recognized term “saponin” includes the plant derived glycosides, comprising generally a sapogenein portion and a sugar moiety, which are produced by many known plant species and which have detergent properties. Saponins are described in, for example, Budvari, ed., The Merck Index, 11^{th} ed. Merck & Co., Inc. Rahway, N.J., 1990, pp. 1328. and Hostettmann, and Marston, Saponins: Chemistry and Pharmacology of Natural Products, 1995, Cambridge University Press, incorporated herein by reference. The saponins of the invention include those which comprise a steroid or a triterpene portion. The saponins of the invention further include those which comprise glucose, galactose pentose and methyl pentose as a sugar moiety. Preferred saponins of the present invention are a sterol glycoside form which is widely distributed in plants. Preferred saponins for use in the present invention are organic emulsifiers derived from Yucca, particularly Yucca schidigera, Yucca valida, or Yucca quijalla. Of the saponin surfactants, those which are nonionic are particularly preferred.

Moreover, improvements to existing methods and compositions are encompassed in the invention described herein, at least in part, by virtue of the fact that the emulsifying agent, saponin compounds, are natural products. As such, they provide novel and improved means of using aqueous-recalcitrant compounds with concurrently diminished hazards and concerns associated with synthetic emulsifiers or concentrating compounds.

In preferred embodiments, the invention includes stable emulsions of at least one aqueous-recalcitrant compound and saponin, wherein the aqueous-recalcitrant compound(s) comprises at least about 50%, 55%, 60%, 65%, 70%, 75%, 80%, 85%, 90%, 95%, or more by weight of the emulsion. In another preferred embodiment, the invention includes stable emulsions of at least two aqueous-recalcitrant compound and saponin, and wherein the aqueous-recalcitrant compounds comprise at least 50%, 55%, 60%, 65%, 70%, 75%, 80%, 85%, 90%, 95%, or more by weight of the emulsion.

In further preferred embodiments, the invention includes stable emulsions of at least one aqueous-recalcitrant compound and saponin, wherein the ratio of saponin to aqueous-recalcitrant compound(s) is 1/34, 1/35, 1/40, 1/45, 1/50, 1/55, 1/60, 1/65, 1/70, 1/75, 1/78, 1/80, 1/85, 1/90, 1/95, 1/100, 1/125, 1/150, 1/175, 1/196, 1/200, or less (mass of saponin/mass of aqueous-recalcitrant compound).

In particularly preferred embodiments, an aqueous-recalcitrant compound which is able to be emulsified by the methods and compositions described herein may be benzyl benzoate, cedryl acetate, cinnamadehyde, L-limonene, D-limonene, or vanillin. The structures of these preferred compounds are set forth below.

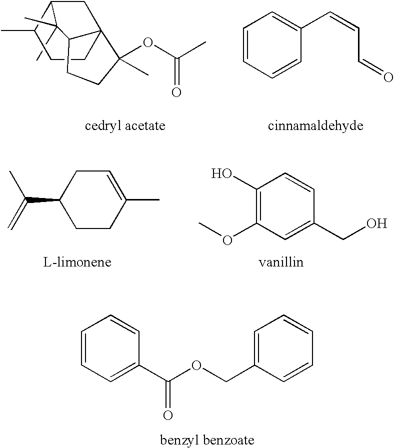

The compositions and methods of the invention described herein can be used to enhance the utility of (i.e., improve) a variety of commercially useful compositions and methods which are recognized in the art and which comprise compounds which are recalcitrant to aqueous solubilization. These improvements are encompassed in the invention described herein, at least in part, by virtue of the fact that the present invention provides methods and compositions to concentrate such compounds in an aqueous solution at higher concentrations than previously possible.

Embodiments of the invention include useful compositions which are stable emulsions and which contain which are recalcitrant to aqueous solubilization in concentrations as high as 80% and 90% (see, e.g., Example 1). Accordingly, a composition of an embodiment of this invention may be prepared as a concentrate for convenient storage and/or transportation as required. Compositions of the present may be adjusted accordingly for large scale (e.g. industrial) utility or for smaller scale (e.g., private) utility. A composition of the invention may require appropriate dilution as needed from a concentrate, or may be manufactured as a “ready-to-apply” composition.

The compositions and methods of the invention described herein can further be used in novel pest control methods and compositions. In an exemplary and preferred embodiment, pest control compositions of the invention comprise saponin as an organic emulsifying agent of D-limonene emulsions and also as a synergistic component (i.e., saponin plays a dual role as an emulsifier and as a synergist). As used herein, the terms “synergist” and “synergistic component” are meant to include compounds and components which can increase the efficacy and/or effectiveness of compositions of the invention. A synergist compound of the invention maximizes, or increases by more than an additive amount, the efficacy and/or effectiveness of compositions of the invention to control pests. For example, the use of saponin as pest control synergist, in combination with other pest control compounds, is described in U.S. Pat. No. 5,639,794, which is incorporated herein by reference. As further used herein, the term synergist includes a compound of the invention which maximizes efficacy or effectiveness of compositions of the invention to control pest organisms, such that a reduction of the amount of compositions of the invention will comprise a pesticidally effective amount.

In general, the emulsions containing high concentrations of aqueous-recalcitrant compounds are made by starting with a preparation that is about a 30%, 40%, 50%, 60%, or 70% by weight emulsion with saponin at a ratio of 1:20 to 1:30 with respect to the organic molecule of interest (for example, 60% organic molecule, 2-3% saponin containing extract (which ranges from about 6-11% saponin by weight) and the balance made up with water (37-38%). Once this stable emulsion is formed, more organic molecule is then added to the original emulsion while agitating to bring the concentration of organic molecule upward to 80-90% w/w. The concentrated emulsions readily re-disperse upon dilution in water to working concentrations of interest. The final concentration of saponin with respect to the organic molecule is easily obtainable at 1:50 or 1:100 w/w, or even less. The shelf life of the concentrated emulsions, either at room temperature (about 25° C.) or at refrigerator temperature (about 4° C.) is at least 1 year, although separation of an emulsion, once stably formed, has not been observed.

For certain applications, it will be desirable to combine terpene compounds in a concentrated emulsion preparation, or to include additional organic compounds that complement the action of the primary organic compound (i.e. the terpene). As an example, in the application of arthropod control, a stable emulsion was prepared containing 40% limonene and 40% benzyl salicylate using purified yucca saponin. The limonene component provides immediate knockdown against the arthropod targets and the benzyl salicylate component provides residual repellency. In another example, a stable emulsion was prepared containing limonene and less than about 0.16% w/w of citric acid. Another stable emulsion was prepared containing 40% limonene and 40% benzyl salicylate and about 0.128% w/w citric acid.

The data provided below clearly demonstrate that stable emulsions containing at least 50% by weight concentrations of organic compounds that are generally considered aqueous recalcitrant can be easily prepared. Of particular interest for use in the preparation of highly concentrated emulsions are the organic compounds cedryl acetate, cinnemaldehyde, and D-limonene, because emulsions containing about 80-90% of the organic compound were accomplished. Additional organic compounds of interest include coniferaldehyde, sinapaldehyde, eugenol, and methyl jasmonate, which did not form stable emulsions when directly added at a desired concentration, but would be expected to form a highly concentrated (at least 50-90% by weight organic compound) stable emulsion by applying the two step mixing method described herein.

The following examples are offered by way of illustration of the present invention, not limitation.

### EXAMPLES

### Example 1

Production of Concentrated Stable Emulsions of Aqueous-Recalcitrant Compounds in Water Using Yucca shidegera Saponin

mulsions of some aqueous-recalcitrant compounds were prepared in water using Yucca shidegera extracts containing about 12-14% saponin, as an emulsifying agent. The saponin preparations were obtained from Danco Natural Products, Pine Valley, Calif., and are sold under the trade names Pure Yucca (about 7-14% saponin and Yucca Ultra (about 10-11% saponin). These preparations had been filtered by the supplier to remove any fibers which could interfere with the emulsification process. The components were combined as follows, using D-limonene as an example.

D-limonene (B. F. Goodrich/Kalama Chemicals, Kalama, Wash.) in the amount of 50 grams was added directly to a clean glass container. To this, 1.6 grams of yucca saponin was added. The glass container was covered tightly and agitated vigorously. A poor, partially opaque solution resulted. This solution was not a stable emulsion. 18.4 grams water was then added such that the total weight of the three components was 70 grams. The mixture was agitated again vigorously in a capped container. A viscous, white and truly opaque emulsion resulted. This was a stable emulsion. To this, another 30 grams limonene was added while mixing to form 100 g final emulsion. The final concentration of D-limonene in the emulsion was 80% by weight. An additional 10 grams of D-limonene can be to bring the final concentration to 90% by weight.

It was determined that this stable emulsion can be repeatedly prepared with a 50:12:1 ratio (weight weight: weight) of D-limonene water: saponin. It was subsequently observed that such emulsions will not undergo phase separation for up to two weeks at ambient temperature. It was also determined that the order in which the formula components are added does not hinder or alter the formation of an emulsion, i.e., order of addition is not important. Furthermore, no two of the three components were able to form an emulsion on their own under the stated conditions. Emulsions were only possible with all three components. The D-limonene emulsion formed by this method could be readily diluted up to eighty-fold in water.

Likewise, other organic compounds can be emulsified by combining about 50 grams of the organic compound with the fraction of saponin indicated in Table 1 and water in a sufficient amount to produce an emulsion upon mixing of the three components. Following formation of the emulsion, the amount of organic compound can be increased as desired.

TABLE 1
Yucca shidegera saponin emulsions of aqueous-recalcitrant compounds

| | | | | Ratio of saponin |
|---|---|---|---|---|
| | | Saponin | Water | to emulsified |
| | Content | content | content | molecule |
| molecule | (by mass) | (by mass) | (by mass) | (by mass) |
| benzyl benzoate | .534 | .010 | .456 | 1/55 |
| cedryl acetate | .794 | .004 | .202 | 1/196 |
| cinnamaldehyde | .899 | .018 | .083 | 1/50 |
| (+) limonene | .690 | .038* | .272 | 1/18* |
| d-limonene | .909 | .012 | .079 | 1/78 |
| vanillin** | .566 | .017 | .418 | 1/34 |

*purified yucca (saponin + sugar moieties) used, therefore saponin content much lower by weight.
**vanillin exists as solid at room temperature, so this was prepared by melting to liquid (>81° C.) to mix liquids, then allowing to cool. Emulsion remains stable at room temperature without recrystalizing).

The emulsions formed in the table set forth above were observed to be stable and, moreover, were readily re-diluted into aqueous solutions. It is worth noting that vanillin is a solid at room termperature. The vanillin emulsion was prepared by first heating vanillin to at least 81° C. in order to melt it, then adding the other components. The resulting vanillin emulsion was stable at room temperature without recrystallizing, i.e., the effective freezing point was lowered in the presence of saponin. In preliminary experiments, stable emulsions were not obtained with coniferaldehyde, sinapaldehyde or eugenol. However, in these experiments, the two step process outlined above was not used and it is possible that obtaining a stable emulsion will be achieved by following the two step process.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporate by reference.

The invention now having been fully described, it will be apparent to one of ordinary skill in the art that many changes and modifications can be made thereto without departing from the spirit or scope of the appended claims.

What is claimed is:

1. A method for preparing a stable emulsion comprising at least 50% concentration by weight of one or more aqueous-recalcitrant organic compounds, said method comprising the steps of:
(a) preparing a first mixture by combining at least 30% by weight of a first aqueous-recalcitrant organic compound with an emulsifier and a solvent,
(b) agitating said first mixture sufficiently to form a first stable emulsion,
(c) preparing a second mixture by adding a second aqueous-recalcitrant organic compound to said first stable emulsion, wherein said second organic compound is the same or different organic compound as said first organic compound, and
(d) agitating said second mixture sufficiently to form a second stable emulsion, wherein said second stable emulsion comprises one or more aqueous-recalcitrant organic compound, whereby a stable emulsion comprising at least 50% concentration by weight of one or more aqueous-recalcitrant organic compounds is prepared.

2. The method according to claim 1, wherein said organic compound is a commercially useful composition.

3. The method according to claim 2, wherein said commercially useful composition is selected from the group consisting of cosmetic composition, cleaning composition, and food additive composition.

4. The method according to claim 3, wherein said commercially useful composition is a pest control composition.

5. A method for preparing a stable emulsion comprising at least 50% concentration by weight of one or more aqueous-recalcitrant organic compounds selected from the group consisting of benzyl benzoate, cedryl acetate, cinnamaldehyde, L-limonene, D-limonene, vanillin and terpinyl acetate, said method comprising the steps of:
(a) preparing a first mixture by combining at least 30% by weight of a first aqueous-recalcitrant organic compound with an emulsifier and a solvent,
(b) agitating said first mixture sufficiently to form a first stable emulsion,
(e) preparing a second mixture by adding a second aqueous-recalcitrant organic compound to said first stable emulsion, wherein said second organic compound is the same or different organic compound as said first organic compound, and
agitating said second mixture sufficiently to form a second stable emulsion, wherein said second stable emulsion comprises one or more aqueous-recalcitrant organic compound, whereby a stable emulsion comprising at least 50% concentration by weight of one or more aqueous-recalcitrant organic compounds is prepared.

6. The method according to claim 1, wherein said emulsifier is a saponin.

7. The method according to claim 1, wherein said saponin is a Yucca shidegera saponin or a Yucca quillaja saponin.

8. An emulsion comprising:
at least 50% by weight of one or more aqueous immiscible organic compounds.

9. An emulsion comprising:
at least 80% by weight of one or more aqueous immiscible organic compounds.

10. An emulsion comprising:
at least 50% by weight of one or more aqueous immiscible organic compounds, wherein said one or more aqueous immiscible organic compounds is selected from the group consisting of benzyl benzoate, cedryl acetate, cinnamaldehyde, L-limonene, D-limonene, vanillin and terpinyl acetate.

11. An aqueous emulsion comprising:
a saponin and at least 50% by weight of one or more aqueous immiscible organic compounds, wherein said one or more aqueous immiscible organic compounds is selected from the group consisting of benzyl benzoate, cedryl acetate, cinnamaldehyde, D-limonene, vanillin and terpinyl acetate.

12. The emulsion according to claim 8 or 9, wherein said emulsion comprises a saponin.

13. The emulsion according to claim 8 or 9, wherein said saponin is a Yucca shidegera saponin or a Yucca quillaja saponin.

14. An emulsion comprising:
at least 80% D-Limonene and a Yucca shidegera saponin.

15. A pest control composition comprising:
at least 80% by weight of a combined mixture of D-Limonene and benzyl salicylate.

16. An aqueous emulsion comprising:
a saponin and at least 50% by weight of benzyl benzoate.

17. An aqueous emulsion comprising:
a saponin and at least 75% by weight of cedryl acetate.

18. An aqueous emulsion comprising:
a saponin and at least 85% by weight of cinnamaldehyde.

19. An aqueous emulsion comprising:
a saponin and at least 90% by weight of D-limonene.

20. An aqueous emulsion comprising:
a saponin and at least 55% by weight of vanillin.
